(19) United States
(12) Patent Application Publication
(10) Pub. No.: US 2005/0267544 A1
(43) Pub. Date: Dec. 1, 2005

(54) Low frequency stimulator provided in a mobile terminal and method for controlling the same

(75) Inventors: Dong-Yul Lee, Dalsco-gu (KR); Jin-Ho Cho, Dalsco-gu (KR); Hee-Joon Park, Gumi-si (KR)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Samsung Electronics Co., Ltd.

(21) Appl. No.: 11/141,020

(22) Filed: Jun. 1, 2005

(30) Foreign Application Priority Data
Jun. 1, 2004 (KR) 2004-39599

(51) Int. Cl. A61N 1/18

(52) U.S. Cl. 607/46; 607/48

(57) ABSTRACT

Disclosed is a low frequency stimulator provided in a mobile terminal, which comprises a high-voltage pulse generator for adjusting a voltage of the mobile terminal to a level suitable for low frequency stimulation; an output controller for outputting a voltage output from the high-voltage pulse generator in a unipolar or bipolar format and controlling the cycle of the output voltage; an electrode section for delivering a stimulation pulse according to the voltage output from the output controller; and a control section for controlling a first switching signal applied to the high-voltage pulse generator to adjust the level of the voltage and controlling a second switching signal applied to the output controller to output a unipolar or bipolar voltage of the controlled cycle.

### PRIORITY

This application claims the benefit under 35 U.S.C. 119(a) of an application entitled “Low Frequency Stimulator Provided in Mobile Terminal and Method for Controlling Same” filed with the Korean Intellectual Property Office on Jun. 1, 2004 and assigned Serial No. 2004-39599, the entire contents of which are incorporated herein by reference.

### BACKGROUND OF THE INVENTION

1. Field of the Invention

The present invention relates to a low frequency stimulator. More particularly, the present invention relates to a low frequency stimulator provided in a mobile terminal and a method for generating low frequency stimulation pulses through the mobile terminal.

2. Description of the Related Art

A low frequency stimulator refers to a neuromuscular stimulation device designed to help rehabilitate injured muscles and control pain by delivering small electrical pulses through pads applied to the skin. The electrical pulses massage and stimulate paralyzed muscles to contract and relax over-excited nerves, thereby relieving chronic or acute pain. The low frequency stimulator can effectively soothe overall bodily fatigue and expedite natural healthy recovery through autonomic nerve stimulation. People suffering from frequent muscle soreness or stiffness due to vigorous physical activities, such as excessive exercise or labor, may need to carry a portable low frequency stimulator. Various types of low frequency stimulators are currently available from a size as small as a TV remote controller to a larger size for use at home or clinics. With growing health care concerns, studies have been actively conducted in many countries to add a health care function to high-end mobile phones. Thus, low frequency stimulators that can operate in combination with mobile phones have become available.

To use such low frequency stimulators, however, users have to carry a separate electrical device that can be connected to a mobile phone. Despite such inconvenience, separate electrical devices in adapter form are generally used for conventional low frequency stimulators because it is not easy to manufacture a low frequency generating circuit device in a small and light design capable of being mounted in a mobile phone. An external adapter can be of a large size. However, an internal adapter mounted in a mobile phone should have a thickness less than 2.5 mm and a size smaller than 3 cm×2 cm. Mutual-induction type boosters that are generally used for low frequency stimulators cannot meet the size requirements for an internal adapter. Furthermore, many additional elements are necessary to form a circuit for changing stimulation pulses and generating both unipolar (unidirectional) and bipolar (bi-directional (+), (−)) pulses. Therefore, it is difficult to provide an internal adapter mounted within a mobile phone.

### SUMMARY OF THE INVENTION

Accordingly, the present invention has been made to solve the above-mentioned problems occurring in the prior art, and an object of the present invention is to provide a mobile terminal comprising a low frequency stimulator.

Another object of the present invention is to provide a method for outputting various types of stimulation pulses from a mobile terminal with a low frequency stimulator.

In accordance with one aspect of the present invention for accomplishing the above objects, there is provided a low frequency stimulator provided in a mobile terminal. The stimulator comprises a high-voltage pulse generator for adjusting a voltage of the mobile terminal to a level suitable for low frequency stimulation; an output controller for outputting a voltage output from the high-voltage pulse generator in unipolar or bipolar and controlling the cycle of the output voltage; an electrode section for delivering a stimulation pulse according to the voltage output from the output controller; and a control section for controlling a first switching signal applied to the high-voltage pulse generator to adjust the level of the voltage and controlling a second switching signal applied to the output controller to output a unipolar or bipolar voltage of the controlled cycle.

In accordance with another aspect of the present invention, there is provided a method for activating a low frequency stimulator provided in a mobile terminal. The method comprises the steps of setting a low frequency stimulation pulse in a low frequency stimulation mode; selecting an electrode section for outputting the set type of low frequency stimulation pulse; when an electrode section formed on the mobile terminal is selected, outputting a voltage corresponding to the set type of stimulation pulse through the electrode section on the mobile terminal; when a pad for low frequency stimulation is selected, determining whether the pad for low frequency stimulation is inserted into an ear jack of the mobile terminal; and when the pad for low frequency stimulation is inserted into the ear jack, outputting a voltage corresponding to the set type of stimulation pulse through an electrode section formed on the pad for low frequency stimulation.

In accordance with still another object of the present invention, there is provided a method for activating a low frequency stimulator provided in a mobile terminal. The method comprises the steps of when a pad for low frequency stimulation is inserted into an ear jack of the mobile terminal, changing the current mode of the mobile terminal to a low frequency stimulation mode; setting a type of low frequency stimulation pulse in the low frequency stimulation mode; and outputting a voltage corresponding to the set type of stimulation pulse through an electrode section formed on the pad for low frequency stimulation.

### BRIEF DESCRIPTION OF THE DRAWINGS

The above and other objects, features and advantages of the present invention will be more apparent from the following detailed description taken in conjunction with the accompanying drawings, in which:

FIG. 1 is a view illustrating a mobile terminal with a built-in low frequency stimulator according to an embodiment of the present invention;

FIG. 2 is a block diagram of the mobile terminal in FIG. 1 according to an embodiment of the present invention;

FIG. 3 illustrates a boost circuit of the high-voltage pulse generator in FIG. 2 according to an embodiment of the present invention;

FIGS. 4A, 4B, and 4C illustrate signal waveforms for explaining the operation of the high-voltage pulse generator in FIG. 3 according to an embodiment of the present invention;

FIG. 5 illustrates an H-bridge circuit of the output controller in FIG. 2 according to an embodiment of the present invention;

FIGS. 6A, 6B, 6C and 6D illustrate signal waveforms for explaining the operation of the output controller in FIG. 5 according to an embodiment of the present invention;

FIG. 7 illustrates an external low frequency stimulator that can be connected to a mobile terminal according to an embodiment of the present invention;

FIG. 8 is a flow chart showing a process of generating a low frequency stimulation pulse in a mobile terminal according to a first embodiment of the present invention;

FIG. 9 is a flow chart showing a process of generating a low frequency stimulation pulse in a mobile terminal according to a second embodiment of the present invention;

FIG. 10 is a flow chart showing how to set a low frequency stimulation pulse in the process of FIGS. 8 and 9 according to an embodiment of the present invention; and

FIGS. 11A to 11D are waveforms of a signal varying according to the stimulation pulse patterns shown in FIG. 10 according to an embodiment of the present invention.

Throughout the drawings, the same elements are designated by the same reference numeral.

### DETAILED DESCRIPTION OF EXEMPLARY EMBODIMENTS

Hereinafter, embodiments of the present invention will be described with reference to the accompanying drawings. In addition, in the following description of the present invention, a detailed description of known functions and configurations incorporated herein will be omitted for conciseness.

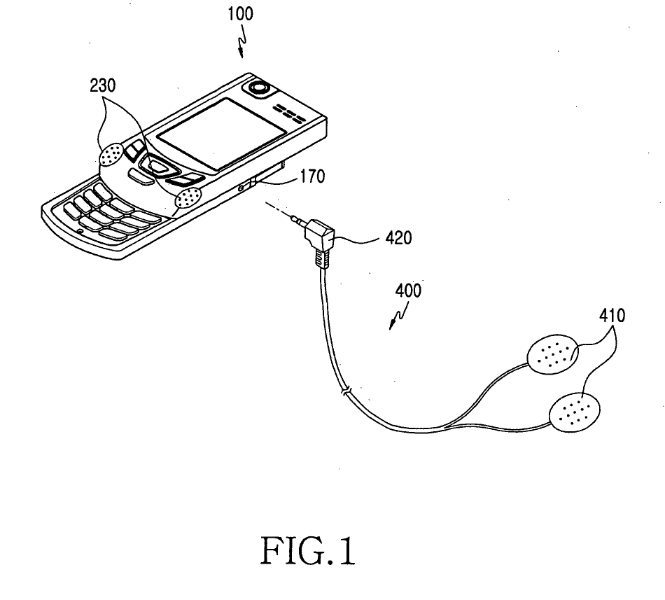

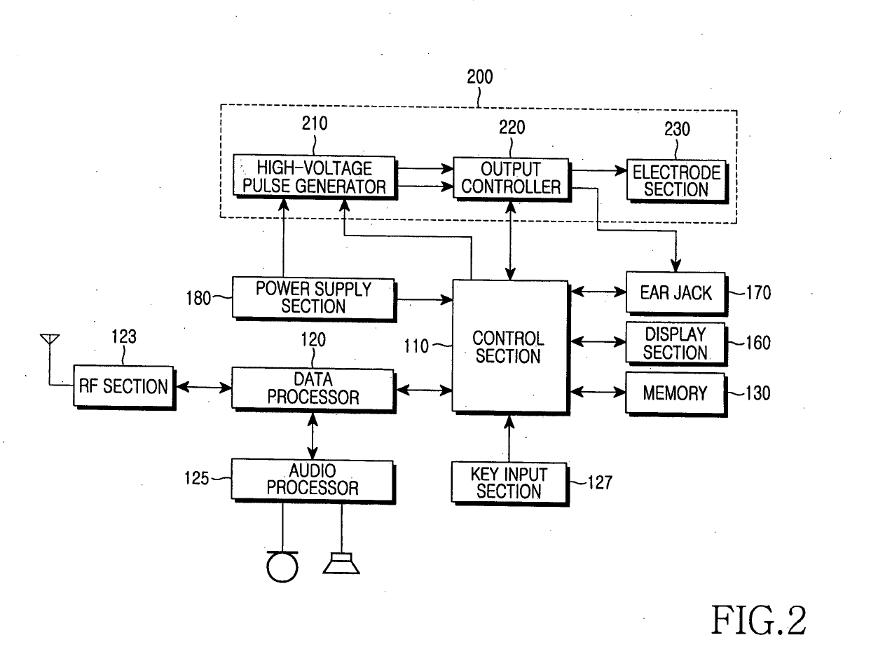

FIG. 1 illustrates a mobile terminal with a built-in low frequency stimulator according to an embodiment of the present invention. Referring to FIG. 1, a mobile terminal 100 includes a low frequency stimulator and an electrode section 230 for delivering a pulse corresponding to a voltage output from the low frequency stimulator. The mobile terminal 100 also has an earphone jack 170 into which a pad 400 for low frequency stimulation can be inserted. The pad 400 includes an electrode section 410 and a plug 420 that can be inserted into the earphone jack 170 of the mobile terminal. When the plug 420 of the pad 400 is inserted into the earphone jack 170 of the mobile terminal, the pulse corresponding to the voltage output from the low frequency stimulator of the mobile terminal is output through the electrode section 410 of the pad 400. The structure of the mobile terminal 100 will be explained in detail with reference to FIG. 2.

Referring to FIG. 1, a radio frequency (RF) section 123 performs a wireless communication function for the mobile terminal. The RF section 123 comprises a RF transmitter (not shown) for performing upward conversion and amplification of the frequency of a transmitted signal, and an RF receiver (not shown) for amplifying a received signal with low noise and performing downward conversion of the frequency of the signal. A data processor 120 comprises a transmitter (not shown) for coding and modulating a signal which is being transmitted and a receiver (not shown) for demodulating and decoding a signal which is being received. The data processor 120 may comprise a modem and a codec. The codec comprises a data codec for processing packet data and an audio codec for processing an audio signal such as a speech signal. An audio processor 125 reproduces an audio signal output from the audio codec of the data processor 120 or transmits an audio signal generated from a microphone to the audio codec of the data processor 120.

A memory 130 may be composed of a program memory and a data memory. The program memory includes programs for controlling general operations of the mobile terminal and those for controlling a low frequency stimulator according to the present invention. The data memory temporarily stores data generated during implementation of the above programs. Also, the memory 130 stores various types of stimulation pulses with different patterns, repetition cycles and intensity levels.

A display section 160 displays messages generated during the implementation of a program under the control of a control section 110 and key data input by a user for a telephone call. When using a liquid crystal display (LCD), the display section 160 may comprise a LCD controller, a memory for storing image data and a LCD device. When the LCD is a touch screen, it can serve as an input section. The display section 160 can display a low frequency stimulation mode and various types of stimulation pulses according to an embodiment of the present invention. A key input section 127 is provided with keys for inputting numbers and characters and function keys for setting up various functions. The key input section 127 may also comprise keys for performing a low frequency stimulation function according to the present invention.

A power supply section 180 supplies a voltage to the mobile terminal. In the low frequency stimulation mode, the power supply section 180 supplies a voltage to a low frequency stimulator 200 provided in the mobile terminal. The earphone jack 170 enables the user to make a voice call using an earphone. According to an embodiment of the present invention, a pad 400 for low frequency stimulation can be inserted into the earphone jack 170 to perform the low frequency stimulation function.

The control section 110 controls the overall operations of the mobile terminal. The control section 110 may comprise the data processor 120. According to an embodiment of the present invention, the control section 110 controls the function of the low frequency stimulator 200. When the user selects a “low frequency stimulation” menu or inserts the pad 400 for low frequency stimulation into the ear jack 170, the control section 110 changes the current mode of the mobile terminal to the low frequency stimulation mode. At this time, the control section 110 can detect the insertion of the pad 400 or an earphone into the earphone jack 170 based on a resistance of the plug inserted into the ear jack 170. Alternatively, the control section 110 can detect the insertion of the pad 400 or the earphone into the earphone jack 170 based on a signal input from an output controller 220. In the low frequency stimulation mode, the control section 110 adjusts the pulse width and repetition cycle of a first switching signal applied to a high-voltage pulse generator 210 of the low frequency stimulator 200 to generate a voltage having a level corresponding to the preset intensity level of the stimulation pulse. Also, the control section 110 adjusts the pulse width of a second switching signal applied to the output controller 220 of the low frequency stimulator 200 to generate a voltage corresponding to the preset pattern of stimulation pulse. Various patterns of stimulation pulse can be set according to selective unipolar or bipolar pulse waveform outputs and pulse repetition cycles. The control section 110 controls the voltage cycle to correspond to the pulse repetition cycle of the preset pattern of stimulation pulse and selectively outputs a unipolar or bipolar pulse waveform. The low frequency stimulator 200 includes an electrode section 230 in addition to the high-voltage pulse generator 210 and the output controller 220. The high-voltage pulse generator 210 adjusts a voltage supplied from the power supply section 180 to a level suitable for low frequency stimulation and outputs the adjusted voltage under the control of the control section 110. The high-voltage pulse generator 210 can boost the single 3V of the mobile terminal 100 to tens to hundreds of volts for the low frequency stimulation.

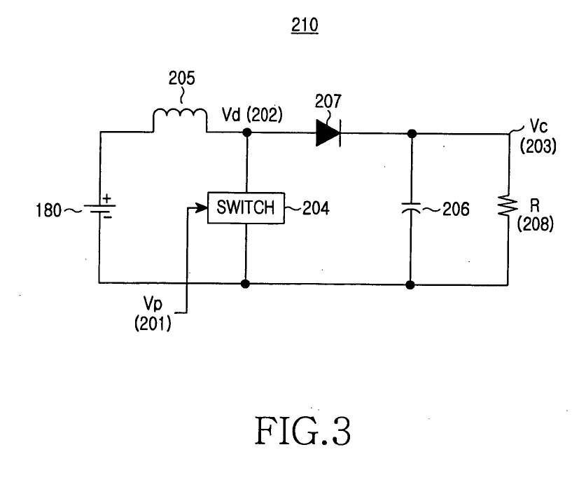

FIG. 3 illustrates a circuit of the high-voltage pulse generator 210. The high-voltage pulse generator 210 employs a boost circuit for boosting a voltage using an inductor voltage. The boost circuit comprises an inductor 205 for inducing a voltage supplied from the power supply section 180, a switch 204 turned on or off by a first switching signal adjusted under the control of the control section 110, a diode 207 for allowing current to flow in one direction and a condenser 206 for storing a voltage for low frequency stimulation. A load resistor 208 which represents a skin contact resistance in the low frequency stimulation mode is not included in the boost circuit of the low frequency stimulator.

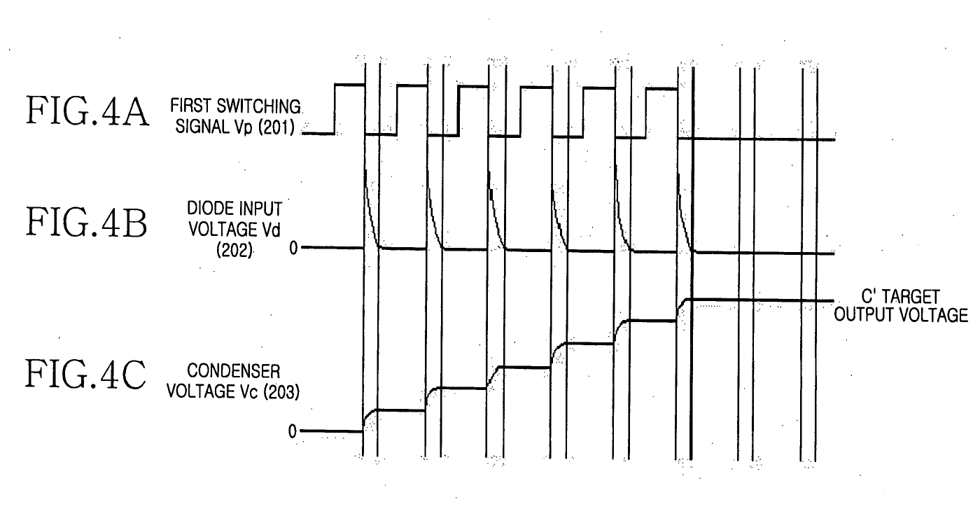

Hereinafter, the boost circuit of the high-voltage pulse generator 210 will be explained in more detail. The control section 110 applies a first switching signal 201 with adjusted pulse width and repetition cycle to the boost circuit in order to output a stimulation pulse of the preset intensity. The switch 204 is turned on or off according to the first switching signal 201 (see FIG. 4A). When the switch 204 is on, the current through the inductor 205 is increasing. At this time, the diode 270 is turned off due to a reverse bias. The voltage across the inductor 205 becomes equal to that supplied from the power supply section 180. When the switch 204 is off, the current through the inductor 205 is decreasing, which results in the change of polarity of the inductor voltage. The inductor voltage is summed with the voltage of the power supply section 180. Accordingly, the diode 207 is forward biased to be turned on again. The voltage across the resistor 208, which is the sum of the voltage across the inductor 205 and the voltage supplied from the power supply section 180, is greater than the voltage of the power supply section 180. The condenser 206 stores the voltage output through the diode 207 and eliminates pulsations from the output voltage 203. When the switch 204 is turned on or off according to the pulse width and repetition cycle of the first switching signal 201 which has been adjusted under the control of the control section 110, the boost circuit of the high-voltage pulse generator 210 generates a voltage of the level corresponding to the preset intensity level of stimulation pulse.

FIG. 4 shows signal waveforms for explaining the operation of the high-voltage pulse generator 210. In FIG. 4, (A) is a waveform of the first switching signal Vp 201. (B) is a waveform of the voltage Vd 202 input to the diode 207 when the first switching signal Vp 201 having the waveform as shown in (A) is input. (C) is a waveform of the voltage Vc 203 input to the condenser 206 when the first switching signal Vp 201 having the waveform as shown in (A) is input. In FIG. 4(C), c′ represents a target output voltage of a level corresponding to the preset intensity level of the stimulation pulse. The output controller 220 can output the voltage output from the high-voltage pulse generator 210 in a unipolar pulse waveform or a bipolar pulse waveform under the control of the control section 110. It is possible to output various patterns of stimulation pulses by controlling the cycle of the voltage output in a unipolar or bipolar pulse waveform. The output controller 220 includes a resistance detector for detecting a resistance value of the earphone or low frequency stimulation pad 400 inserted into the earphone jack 170. The output controller 220 informs the control section 110 of the detected resistance value.

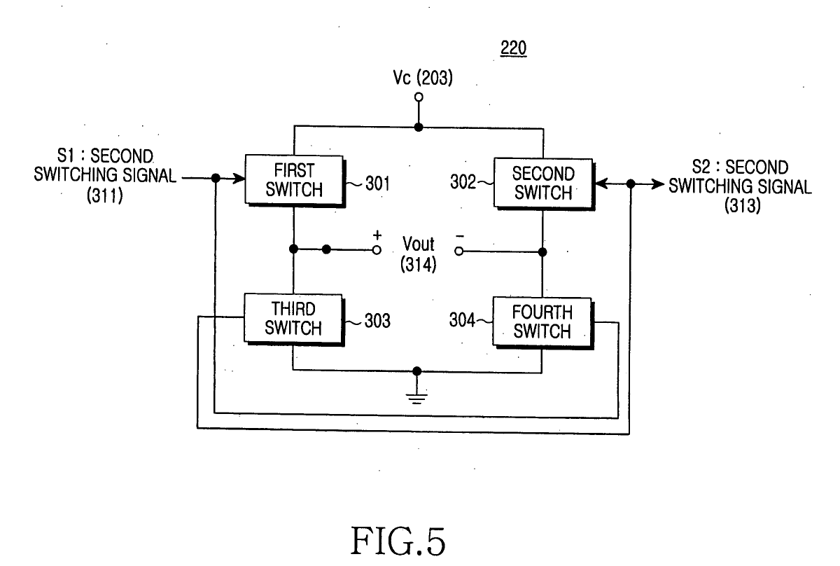

FIG. 5 illustrates a circuit of the output controller 220. Referring to FIG. 5, the output controller 220 uses an H-bridge circuit to generate both unipolar and bipolar outputs. The H-bridge circuit includes four switches 301 to 304 and second switching signals 311 and 313 applied under the control of the control section 110 to control the four switches 301 to 304. Each of the four switches 301 to 304 comprises a transistor and a resistor. The second switching signal S1 311 controls the first and fourth switches 301 and 304, while the second switching signal S2 313 controls the second and third switches 302 and 303. When the second switching signal S1 311 turns on the first and fourth switches 301 and 304 and the second switching signal S2 313 turns on the second and third switches 302 and 303 under the control of the control section 110, the voltage Vc 203 output from the high-voltage pulse generator 210 generates a bipolar pulse waveform output in both forward and backward directions of the voltage output Vout 314. When the second switching signal S1 311 turns on the first and fourth switches 301 and 304, or when the second switching signal S2 313 turns on the second and third switches 302 and 303 under the control of the control section 110, the voltage Vc 203 output from the high-voltage pulse generator 210 generates a unipolar pulse waveform output in either a forward or backward direction of the voltage output Vout 314. The cycle time T of the voltage output in a unipolar or bipolar pulse waveform from the voltage output Vout 314 can be controlled according to the pulse width PW of the second switching signals 311 and 313. Accordingly, it is possible to set and output a pulse pattern having any of a unipolar and bipolar pulse waveforms and pulse repetition cycle T.

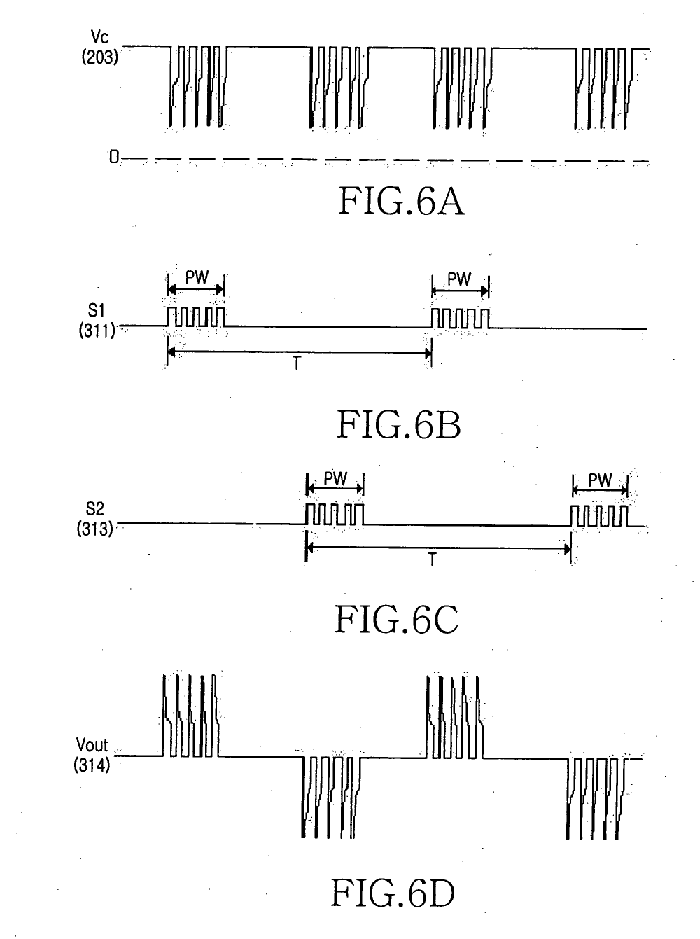

FIG. 6 illustrates signal waveforms for explaining the operation of the output controller 220. In FIG. 6, (A) is a waveform of the voltage Vc 203 output from the high-voltage pulse generator 210. (B) is a waveform of the second switching signal S1 311. (C) is a waveform of the second switching signal S2 313. (d) is a waveform of the output voltage Vout 314 of the output controller 314.

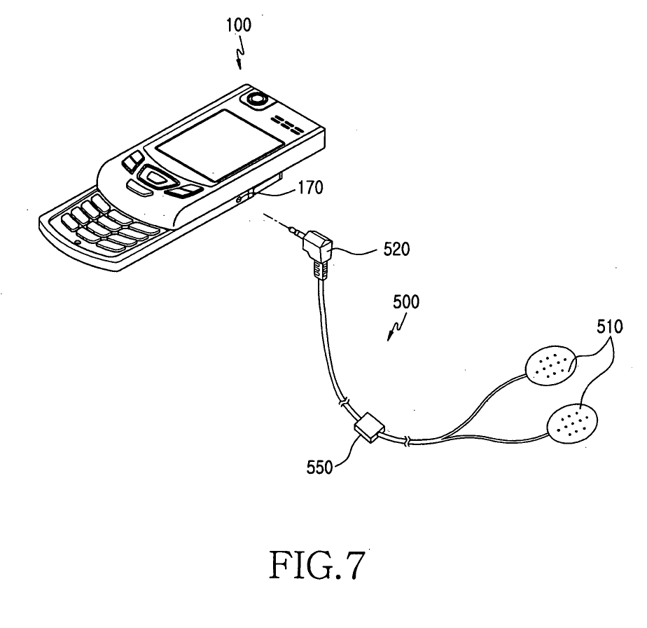

As explained in conjunction with FIGS. 4 to 6, it is possible to control the intensity level of the stimulation pulse by controlling the first switching signal applied to the high-voltage pulse generator 210. A unipolar pulse waveform or a bipolar pulse waveform can be generated according to the second switching signal applied to the output controller 220. The pattern of the stimulation pulse can be determined by adjusting the pulse width PW of the second switching signal and thereby adjusting the repetition cycle T of the stimulation pulse. The electrode section 230 formed on the mobile terminal 100 generates a stimulation pulse corresponding to the voltage output from the output controller 220. When the pad 400 for low frequency stimulation is inserted into the earphone jack 170 of the mobile terminal 100, the stimulation pulse corresponding to the voltage output from the output controller 220 is generated from the electrode section 410 provided on the pad 400. As illustrated in FIG. 1, the low frequency stimulator 200 can operate as either an internal device mounted in the mobile terminal 100 or an external device that can be connected to the mobile terminal 100. FIG. 7 illustrates an external low frequency stimulator that can be connected to the mobile terminal 100 according to an embodiment of the present invention. Referring to FIG. 7, the external low frequency stimulator 500 includes a plug 520 that can be inserted into the earphone jack 170 of the mobile terminal 100, an electrode section 510 for generating a stimulation pulse corresponding to the output voltage and a low frequency stimulation pulse generator 550. The low frequency stimulation pulse generator 550 comprises a high-voltage pulse generator 210 and an output controller 220 which perform the same functions as the high-voltage pulse generator 210 and output controller 220 provided within the mobile terminal 100 in FIG. 2.

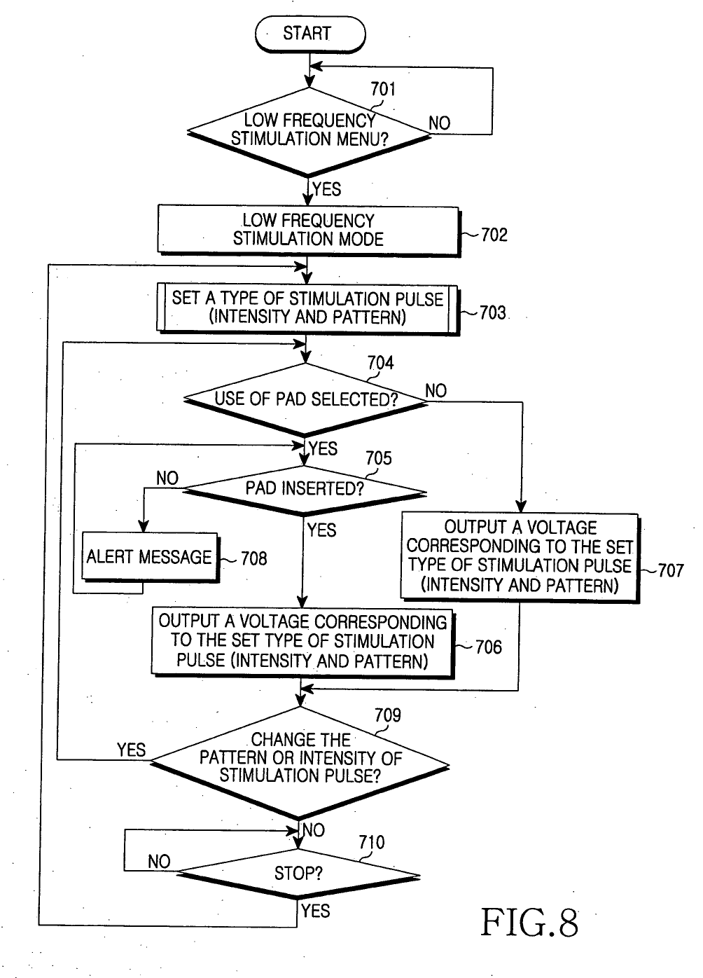

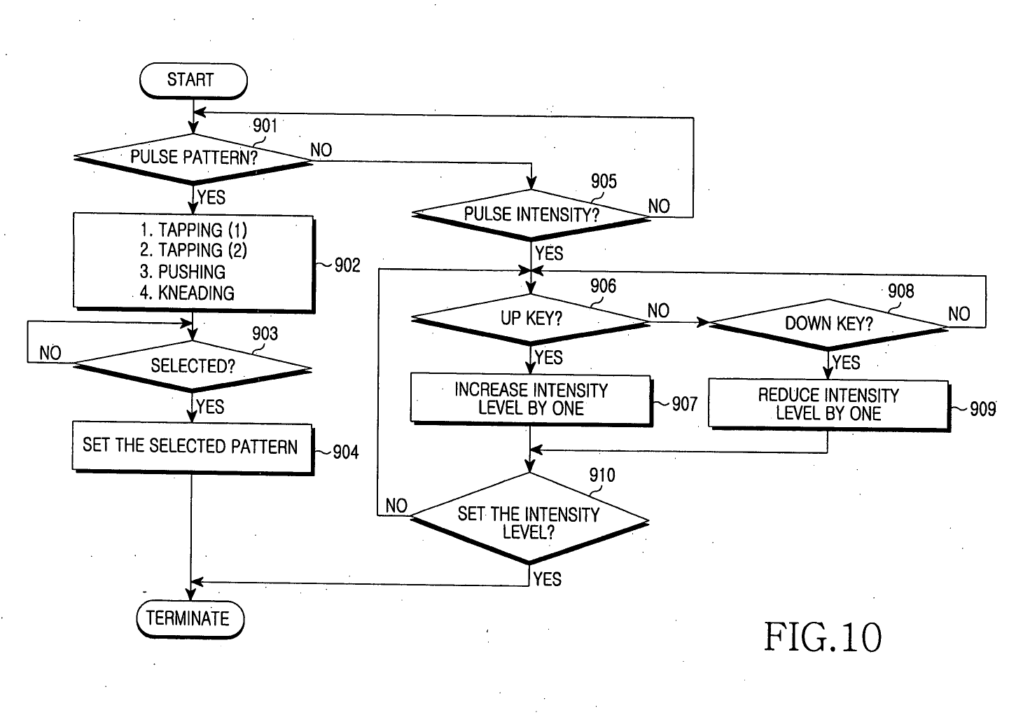

FIG. 8 is a flow chart showing a process of generating a low frequency stimulation pulse in the mobile terminal according to the first embodiment of the present invention. When the user selects a “low frequency stimulation” menu on the mobile terminal, the control section 110 detects the selection at step 701 and changes the current mode of the mobile terminal to a low frequency stimulation mode at step 702. Then the control section 110 proceeds with step 703 to allow the user to set a desired type of stimulation pulse in the low frequency stimulation mode. The process of setting a type of stimulation pulse will be explained in detail with reference to FIG. 10.

When the user sets a type of stimulation pulse and selects the use of a separate pad 400 for low frequency stimulation, the control section 110 detects the selection at step 704 and determines whether the pad 400 for low frequency stimulation is inserted into the earphone jack 170 at step 705. Upon detecting the insertion of the plug 420 of the pad 400 into the earphone jack 170, the output controller 220 detects the resistance value of the inserted plug 420 and informs the control section 110 of the detected resistance value. Then the control section 110 recognizes the insertion of the pad 400 into the earphone jack 170 at step 705 and proceeds with step 706 to generate the set type of stimulation pulse from the electrode section 410 of the pad 400. When the plug of an earphone, rather than the pad 400, is inserted into the earphone jack 170, the output controller 220 detects the resistance value of the inserted plug of the earphone and informs the control section 110 of the detected resistance value. Then the control section 110 recognizes the insertion of the earphone into the earphone jack 170 at step 705 and proceeds with step 708 to generate an alert message or sound. At step 706, the control section 110 supplies a single voltage stored in the power supply section 180 to the high-voltage pulse generator 210. Upon receiving the single voltage of the mobile terminal, the high-voltage pulse generator 210 outputs a voltage of a level corresponding to the intensity level the of stimulation pulse which has been set at step 703 according to the first switching signal 201 adjusted under the control of the control section 110. The voltage output from the high-voltage pulse generator 210 is input to the output controller 220 which will then output the voltage as a unipolar pulse waveform or bipolar pulse waveform according to the second switching signals 311 and 313 applied under the control of the control section 110. The voltage output in unipolar or bipolar pulse waveform is controlled to be output in a cycle corresponding to the preset repetition cycle of stimulation pulse according to the pulse width of the second switching signals 311 and 313. In other words, the voltage is output in the stimulation pulse pattern as set at step 703. The stimulation pulse corresponding to the voltage output from the output controller 220 is delivered through the electrode section 410 of the pad 400 for low frequency stimulation. The user can get a desired neuromuscular stimulation by placing the electrode section 410 on the region of body to be treated. During the treatment with a specific type of stimulation pulse delivered through the electrode section 410, the user may change the type of stimulation pulse. The control section 110 detects the change of the stimulation pulse type at step 709 and proceeds again with step 703. If the user presses a key to stop the low frequency stimulation, the control section 110 will detect the key input at step 710 and will stop the implementation of the low frequency stimulation function accordingly.

After setting a specific type of stimulation pulse, the user may select the output of the stimulation pulse through the electrode section 230 provided on the mobile terminal 100. The control section 110 detects the selection at step 704 and proceeds with step 707 to generate the set type of stimulation pulse through the electrode section 230 of the mobile terminal 100. At step 707, the control section 110 supplies the single voltage stored in the power supply section 180 to the high-voltage pulse generator 210. Upon receiving the single voltage of the mobile terminal, the high-voltage pulse generator 210 outputs a voltage of a level corresponding to the intensity level of stimulation pulse which has been set at step 703 according to the first switching signal 201 adjusted under the control of the control section 110. The voltage output from the high-voltage pulse generator 210 is input to the output controller 220 which will then output the voltage in unipolar pulse waveform or bipolar pulse waveform according to the second switching signals 311 and 313 applied under the control of the control section 110. The voltage output in unipolar or bipolar pulse waveform is controlled to be output in a cycle corresponding to the preset repetition cycle of stimulation pulse according to the pulse width of the second switching signals 311 and 313. In other words, the voltage is output in the stimulation pulse pattern as set at step 703. The stimulation pulse corresponding to the voltage output from the output controller 220 is delivered through the electrode section 230 formed on the mobile terminal 100. The user can get a desired neuromuscular stimulation by holding the electrode section 230 of the mobile terminal 100 or placing the electrode section 230 on the region of body to be treated. During the treatment with a specific type of stimulation pulse delivered through the electrode section 230, the user may change the type of stimulation pulse. The control section 110 detects the change of the stimulation pulse type at step 709 and proceeds again with step 703. If the user presses a key to stop the low frequency stimulation, the control section 110 will detect the key input at step 710 and will stop the implementation of the low frequency stimulation. If an incoming call is received in the low frequency stimulation mode, the control section 110 will detect the incoming call signal and will change the current mode to a call mode. Upon completion of a telephone call in the call mode, the control section 110 will automatically convert into the low frequency stimulation mode to continue implementation of the low frequency stimulation. Alternatively, when an incoming call is received in the low frequency stimulation mode, the control section 110 may activate the call mode while maintaining the low frequency stimulation mode.

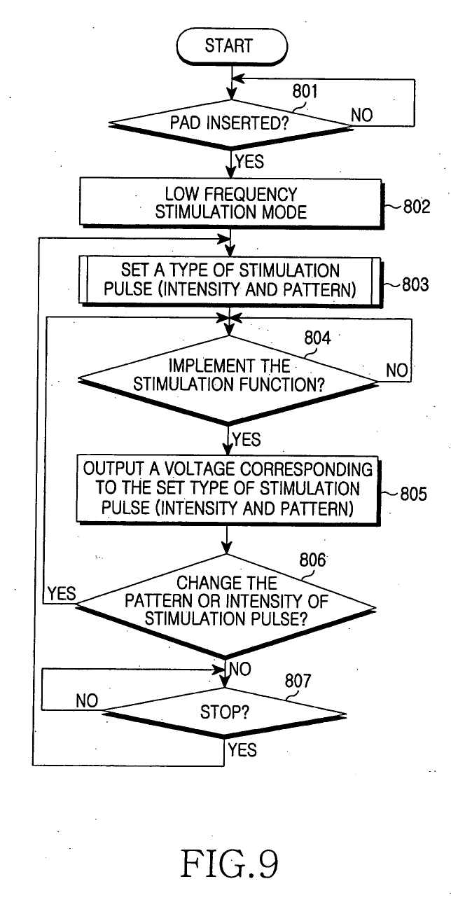

FIG. 9 is a flow chart showing a process of generating a low frequency stimulation pulse according to the second embodiment of the present invention. The process will be explained in detail with reference to FIGS. 1 to 6. When the pad 400 for low frequency stimulation is inserted into the earphone jack 170 of the mobile terminal 100, the output controller 220 detects the resistance value of the plug inserted into the earphone jack 170 and informs the control section 110 of the detected resistance value. Then the control section 110 recognizes the insertion of the pad 400 into the earphone jack 170 at step 801 and proceeds with step 802 to change the current mode of the mobile terminal 100 to the low frequency stimulation mode. In the low frequency stimulation mode, the control section proceeds with step 803 to allow the user to set a desired type of stimulation pulse. The process of setting a type of stimulation pulse will be explained in detail with reference to FIG. 10. When the user sets a specific type of stimulation pulse and selects the implementation of the low frequency stimulation function, the control section 110 detects the selection at step 804 and proceeds with step 805 to generate the set type of stimulation pulse through the electrode section 410 of the pad 400 for low frequency stimulation. At step 805, the control section 110 supplies a single voltage of the mobile terminal stored in the power supply section 180 to the high-voltage pulse generator 210. Upon receiving the single voltage of the mobile terminal, the high-voltage pulse generator 210 outputs a voltage of a level corresponding to the intensity level of stimulation pulse which has been set at step 803 according to the first switching signal 201 adjusted under the control of the control section 110. The voltage output from the high-voltage pulse generator 210 is input to the output controller 220 which will then output the voltage in unipolar pulse waveform or bipolar pulse waveform according to the second switching signals 311 and 313 applied under the control of the control section 110. The voltage output in unipolar or bipolar pulse waveform is controlled to be output in a cycle corresponding to the preset repetition cycle of the stimulation pulse according to the pulse width of the second switching signals 311 and 313. In other words, the voltage is output in the stimulation pulse pattern as set at step 803. The stimulation pulse corresponding to the voltage output from the output controller 220 is delivered through the electrode section 410 of the pad 400 for low frequency stimulation. The user can get a desired neuromuscular stimulation by placing the electrode section 410 on the region of body to be treated. During the treatment with a specific type of stimulation pulse delivered through the electrode section 410, the user may change the type of stimulation pulse. The control section 110 detects the change of the stimulation pulse type at step 806 and proceeds again with step 803. If the user presses a key to stop the low frequency stimulation, the control section 110 will detect the key input at step 807 and will stop the implementation of the low frequency stimulation function. If an incoming call is received in the low frequency stimulation mode, the control section 110 will detect the incoming call signal and will change the current mode to the call mode. Upon completion of a telephone call in the call mode, the control section 110 will automatically enter the low frequency stimulation mode to continue implementation of the low frequency stimulation. Alternatively, when an incoming call is received in the low frequency stimulation mode, the control section 110 may activate the call mode while maintaining the low frequency stimulation mode.

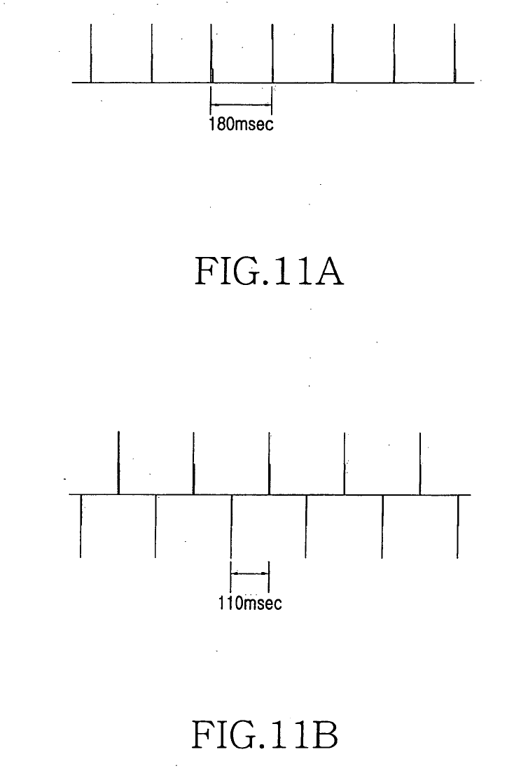

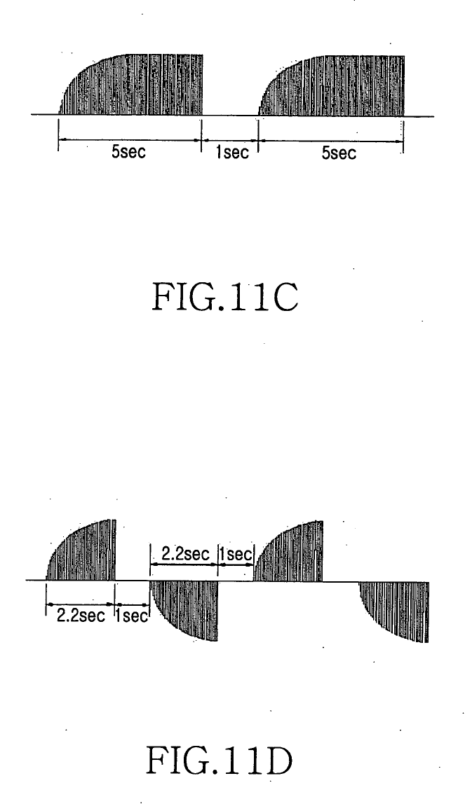

FIG. 10 is a flow chart showing how to set a specific type of stimulation pulse in the process of FIG. 8 or 9. FIGS. 11A to 11D are waveforms of a signal varying according to the stimulation pulse patterns in FIG. 10. Types of stimulation pulses in the present invention refer to the patterns and intensity levels of stimulation pulses. It is assumed that a type of stimulation pulse can be set based on 13 levels of intensity and 4 different patterns. Hereinafter, the process of setting a specific type of stimulation pulse will be explained with reference to FIGS. 1 to 6, 8 and 9. When the user selects a “type of stimulation pulse” menu in the low frequency stimulation mode, the control section 110 detects the selection and displays two items “pulse pattern” and “pulse intensity”. When the user selects the “pulse pattern” item, the control section detects the selection at step 901 and changes the current mode to a pulse pattern setting mode. The control section 110 then displays available patterns at step 902. When the user selects “tapping (1)” from the displayed patterns, the control section 110 detects the selection at step 903 and proceeds with step 904 to set the stimulation pulse in tapping (1) pattern. The tapping (1) pattern has a unipolar pulse waveform and a repetition cycle of 180 ms. FIG. 11A shows a signal waveform of the tapping (1) pattern of stimulation pulse output at step 706 in FIG. 8 and step 805 in FIG. 9. When the user selects “tapping (2)” from the displayed patterns, the control section 110 detects the selection at step 903 and proceeds with step 904 to set the stimulation pulse in tapping (2) pattern. The tapping (2) pattern has a bipolar pulse waveform and a repetition cycle of 110 ms. FIG. 11B shows a signal waveform of the tapping (2) pattern of stimulation pulse output at step 706 in FIG. 8 and step 805 in FIG. 9. When the user selects “pushing” from the displayed patterns, the control section 110 detects the selection at step 903 and proceeds with step 904 to set the stimulation pulse in pushing pattern. The pushing pattern has a unipolar pulse waveform and a repetition cycle of 30 ms. FIG. 11C shows a signal waveform of the pushing pattern of stimulation pulse output at step 706 in FIG. 8 and step 805 in FIG. 9. When the user selects “kneading” from the displayed patterns, the control section 110 detects the selection at step 903 and proceeds with step 904 to set the stimulation pulse in kneading pattern. The pushing pattern has a bipolar pulse waveform and a repetition cycle of 30 ms. FIG. 11D shows a signal waveform of the kneading pattern of stimulation pulse output at step 706 in FIG. 8 and step 805 in FIG. 9. The user can set a desired pattern of stimulation pulse by selecting one of the four displayed patterns or pressing a mode key provided on the key input section 127. The mode key can change the pulse pattern when pressed for a period shorter than a predetermined period of time. When the mode key is pressed longer, the low frequency stimulation mode will be terminated.

When the user selects the “pulse intensity” item, the control section detects the selection at step 905 and initiates a pulse intensity setting mode. When the user presses an upward direction key in the pulse intensity setting mode, the control section 110 detects the key input at step 906 and proceeds with step 907 to increase the current intensity level by one. When the user presses a downward direction key in the same mode, the control section 110 detects the key input at step 908 and proceed with step 909 to reduce the intensity level by one. Assuming that the stimulation pulse intensity can be set from 13 levels, voltages corresponding to the 13 levels of intensity are indicated in Table 1.

TABLE 1

| | | | | | | | | | | | | | |
|---|---|---|---|---|---|---|---|---|---|---|---|---|---|
| Level | 1 | 2 | 3 | 4 | 5 | 6 | 7 | 8 | 9 | 10 | 11 | 12 | 13 |
| Voltage | 30 V | 42 V | 50 V | 56 V | 62 V | 67 V | 72 V | 76 V | 79 V | 83 V | 86 V | 89 V | 91 V |

The user can set a desired level of pulse intensity using the up and down keys. It is possible to change the intensity level even during the treatment with a selected pattern of stimulation pulses. When the user completes the adjustment of the intensity level using the up and down keys, the control section 110 detects the adjusted intensity level at step 910 and generates the stimulation pulses at the adjusted intensity level.

Although certain embodiments of the present invention have been described for illustrative purposes, those skilled in the art will appreciate that various modifications, additions and substitutions are possible, without departing from the scope and spirit of the invention as disclosed in the accompanying claims, including the full scope of equivalents thereof.

1. A low frequency stimulator provided in a mobile terminal, which comprises:
a high-voltage pulse generator for adjusting a voltage of the mobile terminal to a level suitable for low frequency stimulation;
an output controller for outputting a voltage output from the high-voltage pulse generator in a unipolar or bipolar format and controlling the cycle of the output voltage;
an electrode section for delivering a stimulation pulse according to the voltage output from the output controller; and
a control section for controlling a first switching signal applied to the high-voltage pulse generator to adjust the level of the voltage and controlling a second switching signal applied to the output controller to output a unipolar or bipolar voltage of the controlled cycle.

2. The low frequency stimulator as claimed in claim 1, wherein said high-voltage pulse generator uses a boost circuit to output a voltage of a level suitable for low frequency stimulation.

3. The low frequency stimulator as claimed in claim 1, wherein said output controller uses an H-bridge circuit to output unipolar and bipolar voltages of a controlled cycle.

4. The low frequency stimulator as claimed in claim 1, wherein said electrode section is formed on the mobile terminal.

5. The low frequency stimulator as claimed in claim 1, wherein said electrode section is formed on a pad for low frequency stimulation which can be inserted into an ear jack of the mobile terminal.

6. The low frequency stimulator as claimed in claim 1, wherein said output controller determines whether the pad with the electrode section or an earphone is inserted into the ear jack of the mobile terminal based on a resistance value of a plug inserted into the ear jack.

7. The low frequency stimulator as claimed in claim 5, wherein said output controller determines whether the pad with the electrode section or an earphone is inserted into the ear jack of the mobile terminal based on a resistance value of a plug inserted into the ear jack.

8. A method for activating a low frequency stimulator provided in a mobile terminal, which comprises the steps of:
setting a low frequency stimulation pulse in a low frequency stimulation mode;
selecting an electrode section for outputting the set type of low frequency stimulation pulse;
when an electrode section formed on the mobile terminal is selected, outputting a voltage corresponding to the set type of stimulation pulse through the electrode section on the mobile terminal;
when a pad for low frequency stimulation is selected, determining whether the pad for low frequency stimulation is inserted into an ear jack of the mobile terminal; and
when the pad for low frequency stimulation is inserted into the ear jack, outputting a voltage corresponding to the set type of stimulation pulse through an electrode section formed on the pad for low frequency stimulation.

9. The method as claimed in claim 8, further comprising the step of:
when said pad for low frequency stimulation is not inserted into the ear jack, generating an alert message or sound.

10. A method for activating a low frequency stimulator provided in a mobile terminal, which comprises the steps of:
when a pad for low frequency stimulation is inserted into an ear jack of the mobile terminal, changing the current mode of the mobile terminal to a low frequency stimulation mode;
setting a type of low frequency stimulation pulse in the low frequency stimulation mode; and
outputting a voltage corresponding to the set type of stimulation pulse through an electrode section formed on the pad for low frequency stimulation.

11. The method as claimed in claim 8, wherein said step of setting a type of low frequency stimulation pulse comprises:
when a “pulse pattern” item is selected from a stimulation pulse menu, initiating a pulse pattern setting mode;
setting a desired pulse pattern in the pulse pattern setting mode;
when a pulse intensity item is selected from the stimulation pulse menu, initiating a pulse intensity setting mode;
when an upward direction key is pressed, increasing the current intensity level of stimulation pulse by one; and
when a downward direction key is pressed, reducing the current intensity level of stimulation pulse by one.

12. The method as claimed in claim 10, wherein said step of setting a type of low frequency stimulation pulse includes:
when a pulse pattern item is selected from a stimulation pulse menu, initiating a pulse pattern setting mode;
setting a desired pulse pattern in the pulse pattern setting mode;
when a pulse intensity item is selected from the type of stimulation pulse menu, initiating a pulse intensity setting mode;
when an upward direction key is pressed, increasing the current intensity level of stimulation pulse by one; and
when a downward direction key is pressed, reducing the current intensity level of stimulation pulse by one.

13. The method as claimed in claim 12, wherein pulse patterns that can be set in the pulse pattern setting mode comprise first tapping, second tapping, pushing and kneading.

14. The method as claimed in claim 8, further comprising the step of:
when an incoming call is received in the low frequency stimulation mode, changing the mode to a call mode; and
when a telephone call in the call mode is finished, automatically converting into the low frequency stimulation mode.

15. The method as claimed in claim 10, further comprising the step of:
when an incoming call is received in the low frequency stimulation mode, changing the mode to a call mode; and
when a telephone call in the call mode is finished, automatically converting into the low frequency stimulation mode.
